(19) United States
(12) Patent Application Publication
(10) Pub. No.: US 2026/0287545 A1
(43) Pub. Date: Sep. 24, 2026

(54) FIELD EFFECT TRANSISTOR AND METHOD FOR MANUFACTURING SAME

(71) Applicant: LG ELECTRONICS INC., Seoul (KR)

(72) Inventors: Eunhwa CHOI, Seoul (KR); Yunguk JANG, Seoul (KR); Wanseop CHOI, Seoul (KR); Oedong KIM, Seoul (KR)

(73) Assignee: LG ELECTRONICS INC., Seoul (KR)

(21) Appl. No.: 19/475,585

(22) PCT Filed: Apr. 21, 2023
(86) PCT No.: PCT/KR2023/005474

§ 371 Date: Oct. 15, 2025

(51) Int. Cl. G01N 27/414; G01N 33/543

(52) U.S. Cl. CPC G01N 27/4145; G01N 33/5438

(57) ABSTRACT

The present disclosure relates to a field effect transistor, which can be applied to the field of device technology capable of detecting biomaterials and can detect, for example, biochemical molecules, and to a method for manufacturing same. The present disclosure may comprise: a substrate including a semiconductor; an oxide layer positioned on the substrate; a source electrode and a drain electrode positioned to be spaced apart from each other in a first direction of the oxide layer; a first gate electrode and a second gate electrode positioned to be spaced apart from each other in a second direction of the oxide layer; a transducer positioned to connect the source electrode and the drain electrode to each other; a cationic buffer that is at least partially coupled to the transducer and provides transistor characteristics; a bioreceptor positioned on the transducer; and a linker positioned between the transducer and bioreceptor.

### TECHNICAL FIELD

The present disclosure relates to a field of device technology for detecting biomaterials, and relates to, for example, a field effect transistor for detecting biochemical molecules and a method of manufacturing the same.

### BACKGROUND

A biofield effect transistor (bioFET) is a device that detects biochemical molecules, and is also called a bioFET. The bioFET may refer to a device that enable high-sensitivity measurement of biochemical molecules.

In general, a transfer curve of a field effect transistor does not appear as a curve when there is no gating effect. That is, in this case, even if a gate voltage Vg is applied, there is no change in a signal Ids, and thus the field effect transistor may not operate.

In general, it may be difficult to obtain a gating effect in air or gas phase in bioFETs. In a typical bioFET, gating occurs when water (or a buffer) connects a gate electrode and a transducer, but in a gas phase, it may be difficult to induce gating between electrodes unless a high voltage is applied to the gate electrode.

When a high voltage is applied to the gate electrode, the stability of the device may be compromised, and problems such as signal abnormalities in the device may occur.

Therefore, a solution to these problems is required.

### DISCLOSURE

### Technical Problem

An object of the present disclosure is to provide a field effect transistor and a method of manufacturing the same, which may detect a small change on a surface of a device, such as a change in surface potential due to binding of biomolecules or a change in pH, as a large current change through a gating effect.

An object of the present disclosure is to provide a field effect transistor and a method of manufacturing the same, which may increase transconductance through gating induction under gaseous conditions without applying a high voltage.

An object of the present disclosure is to provide a field effect transistor and a method of manufacturing the same, which may effectively transmit a charge change caused by gaseous molecules such as olfactory receptors to a transducer.

### Technical Solution

According to a first aspect, a field effect transistor includes a substrate including a semiconductor, an oxide layer located on the substrate, a source electrode and a drain electrode that are located spaced apart from each other in a first direction of the oxide layer, a first gate electrode and a second gate electrode that are located spaced apart from each other in a second direction of the oxide layer, a transducer located to connect the source electrode and the drain electrode, a zwitterionic buffer that is at least partly coupled to the transducer and provides transistor characteristics, a bioreceptor located on the transducer, and a linker located between the transducer and the bioreceptor.

According to an exemplary embodiment, the transducer may include a graphene layer.

According to an exemplary embodiment, the transducer may form a channel between the source electrode and the drain electrode.

According to an exemplary embodiment, the field effect transistor may include an alignment medium which is located between the bioreceptor and the linker.

According to an exemplary embodiment, the alignment medium may be coupled such that the bioreceptor and the linker are arranged in series.

According to an exemplary embodiment, the zwitterionic buffer may include hydroxyethyl piperazine ethane sulfonicacid (HEPES).

According to an exemplary embodiment, the zwitterionic buffer may provide a V-shaped transfer curve.

According to a second aspect, a field effect transistor includes a substrate including a semiconductor, an oxide layer located on the substrate, a source electrode and a drain electrode that are located spaced apart from each other in a first direction of the oxide layer, a first gate electrode and a second gate electrode that are located spaced apart from each other in a second direction of the oxide layer, a transducer located to connect the source electrode and the drain electrode, and a detection layer located on the transducer, wherein the detection layer includes a bioreceptor located on the transducer, and a zwitterionic buffer that provides transistor characteristics to the transducer.

According to an exemplary embodiment, the field effect transistor may further include a linker located between the transducer and the bioreceptor.

According to an exemplary embodiment, the field effect transistor may further include an insulating layer that partially exposes upper surfaces of the source electrode and the drain electrode.

According to a third aspect, a method of manufacturing a field effect transistor includes manufacturing a base structure in which a source electrode and a drain electrode, located spaced apart from each other in a first direction, are arranged on an oxide layer located on a substrate including a semiconductor and a transductor is provided to connect the source electrode and the drain electrode, immobilizing a linker on a transducer of the base structure, immobilizing a bioreceptor on the linker, and applying a zwitterionic buffer that is coupled to at least a portion of the transducer to provide transistor characteristics.

According to an exemplary embodiment, Cadaverine may be coupled to the linker.

According to an exemplary embodiment, the immobilizing of the bioreceptor may include adding an alignment medium coupled to Cadaverine.

### Advantageous Effects

According to an embodiment of the present disclosure, the following effects are obtained.

First, a field effect transistor (FET) (bioFET) according to an embodiment of the present disclosure may advantageously detect a small change above a device surface, such as a change in surface potential due to binding between the bioreceptor 310 and a biomolecule or a change in pH, to a large current change through a gating effect.

The FET (bioFET) according to an embodiment of the present disclosure described above may increase transconductance through gating induction under a gaseous condition without applying a high voltage.

The FET (bioFET) according to an embodiment of the present disclosure may effectively transmit a charge change caused by gaseous molecules such as olfactory receptors to a transducer.

According to another embodiment of the present disclosure, there are additional technical effects not mentioned herein. A person skilled in the art may understand the full intent of the specification and drawings.

### BRIEF DESCRIPTION OF THE DRAWINGS

FIG. 1 is a schematic perspective view showing a field effect transistor (FET) according to an embodiment of the present disclosure.

FIG. 2 is a conceptual diagram showing an example of a detection layer of a FET according to an embodiment of the present disclosure.

FIG. 3 is a conceptual diagram showing another example of a detection layer of a FET according to an embodiment of the present disclosure.

FIG. 4 is a molecular structure diagram of PSE, which is an example of a detection layer of an FET according to an embodiment of the present disclosure.

FIG. 5 is a molecular structure diagram of HEPES, which is an example of a zwitterionic buffer in a detection layer of a field effect transistor according to an embodiment of the present disclosure.

FIG. 6 is a graph showing a transfer curve of an FET in a general case.

FIG. 7 is a graph showing a transfer curve of an FET according to an embodiment of the present disclosure.

FIG. 8 is a graph showing a transfer curve of an FET over time in a general case.

FIG. 9 is a graph showing a transfer curve of an FET over time according to an embodiment of the present disclosure.

FIGS. 10 to 20 are cross-sectional views showing a manufacturing process of an FET according to an embodiment of the present disclosure.

FIG. 21 and FIG. 22 are flowcharts showing a manufacturing process of a detection layer of an FET according to an embodiment of the present disclosure.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts, and redundant description thereof will be omitted. As used herein, the suffixes “module” and “unit” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification.

Furthermore, although the drawings are separately described for simplicity, embodiments implemented by combining at least two or more drawings are also within the scope of the present disclosure.

In addition, when an element such as a layer, region or module is described as being “on” another element, it is to be understood that the element may be directly on the other element or there may be an intermediate element between them.

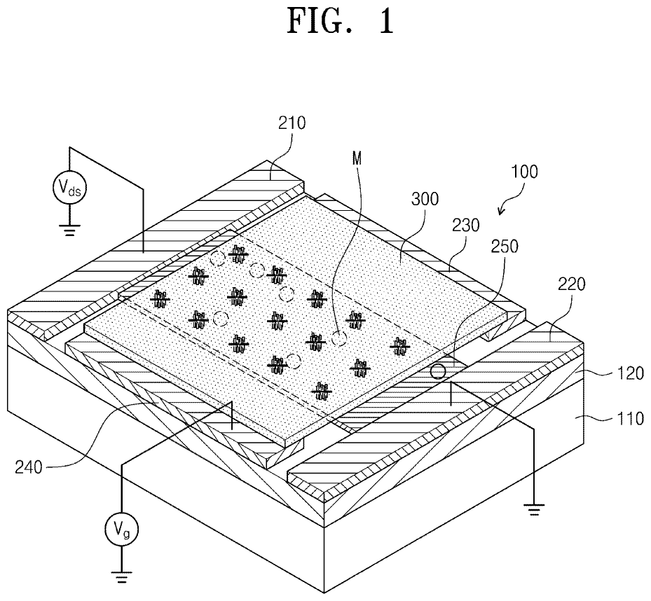

FIG. 1 is a schematic perspective view showing a field effect transistor (FET) according to an embodiment of the present disclosure.

Referring to FIG. 1, a field effect transistor (FET) 100 according to an embodiment of the present disclosure may basically have a metal-oxide-semiconductor (MOS) structure. That is, the FET 100 according to an embodiment of the present disclosure may have a MOSFET structure.

The FET 100 according to an embodiment of the present disclosure may include a substrate 110 including a semiconductor, an oxide layer 120 located on the substrate 110, a source electrode 210 and a drain electrode 220 that are spaced apart from each other in a first direction of the oxide layer 120, and a first gate electrode 230 and a second gate electrode 240 that are spaced apart from each other in a second direction of the oxide layer 120.

The substrate 110 may include a silicon (Si) semiconductor. For example, the substrate 110 may be made of a silicon wafer. The oxide layer 120 may include silicon oxide (SiO2). For example, the oxide layer 120 may be a silicon oxide layer.

Referring to FIG. 1, the source electrode 210 and the drain electrode 220 may be located at both ends of one side of the oxide layer 120 or at positions close to the both ends. Accordingly, the first gate electrode 230 and the second gate electrode 240 may be located at opposite ends of the other side of the oxide layer 120 or at positions close to the opposite ends.

The FET 100 may include a transducer 250 located to connect the source electrode 210 and the drain electrode 220. The transducer 250 may define a channel between the source electrode 210 and the drain electrode 220.

The transducer 250 may include a graphene layer. For example, the transducer 250 may be formed of a graphene layer. The transducer 250 may be located parallel to the first gate electrode 230 and the second gate electrode 240.

In FIG. 1, the transducer 250 is illustrated as being spaced apart from the first gate electrode 230 and the second gate electrode 240 by a certain distance, but the transducer 250 may also be located close to the first gate electrode 230 and the second gate electrode 240.

A detection layer 300 may be located on the transducer 250. The detection layer 300 may detect biochemical molecules M with high sensitivity.

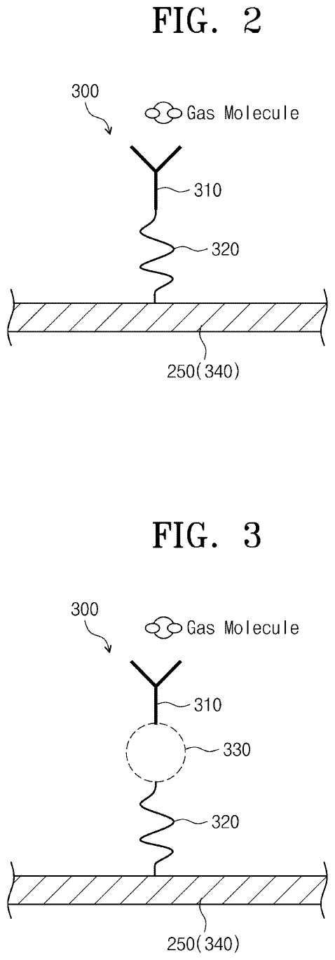

FIG. 2 is a conceptual diagram showing an example of a detection layer of a FET according to an embodiment of the present disclosure. FIG. 3 is a conceptual diagram showing another example of a detection layer of a FET according to an embodiment of the present disclosure.

Referring to FIG. 2, the detection layer 300 may include a zwitterionic buffer 340 that is at least partially coupled to the transducer 250 to provide the characteristics of a transistor, a bioreceptor 310 located on the transducer 250, and a linker 320 located between the transducer 250 and the bioreceptor 310. The linker 320 may provide a connection structure between the transducer 250 and the bioreceptor 310.

The bioreceptor 310 is a biological sensing material. Afield effect transistor according to an embodiment of the present disclosure may change a small change above a surface, such as a change in surface potential due to binding between the bioreceptor 310 and a biomolecule or a change in pH, to a large current change through a gating effect.

The field effect transistor according to an embodiment of the present disclosure may detect a gas molecule M in a gas phase without a buffer liquid with high sensitivity by using the detection layer 300 including the bioreceptor 310.

For example, even if the molecule is not provided in a liquid form, the gas molecule M may be collected in the bioreceptor 310 in the bioreceptor 310 and delivered to the transducer 250 through the linker 320 to cause a gating effect.

The zwitterionic buffer 340 may include hydroxyethyl piperazine ethane sulfonicacid (HEPES). At least a portion of the zwitterionic buffer 340 may be in contact with the transducer 250. For example, the zwitterionic buffer 340 may be applied onto the transducer 250.

The zwitterionic buffer 340 may maintain the structure and function of the bioreceptor 310.

As an exemplary embodiment, the zwitterionic buffer 340 may provide a V-shaped transfer curve to the FET 100. This will be described below in more detail.

Referring to FIG. 3, as an exemplary embodiment, an alignment medium 330 may be included in the detection layer 300. For example, the alignment medium 330 may be located between the bioreceptor 310 and the linker 320.

The alignment medium 330 may be coupled such that the bioreceptor 310 and the linker 320 are arranged in series. In other words, the alignment medium 330 may function as connection such that the bioreceptor 310 and the linker 320 may be be arranged linearly.

FIG. 2 is a molecular structure diagram of PSE, which is an example of a detection layer of an FET according to an embodiment of the present disclosure. FIG. 5 is a molecular structure diagram of HEPES, which is an example of a zwitterionic buffer in a detection layer of a field effect transistor according to an embodiment of the present disclosure.

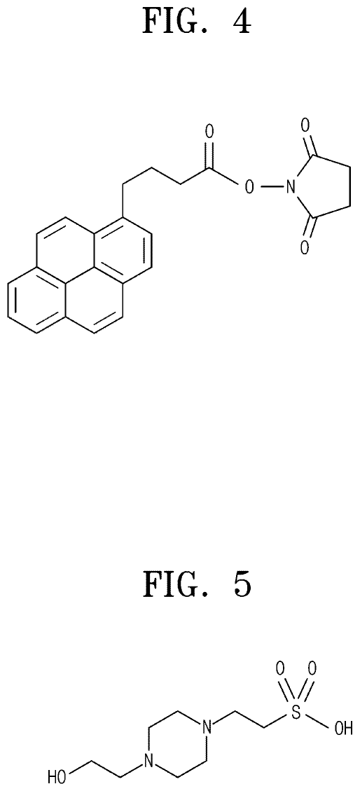

FIG. 4 illustrates a 1-pyrenebutyric acid N-hydroxysuccinimide ester (PSE) linker as an example of the linker 320 included in the detection layer 300. The PSE linker 320 may immobilize the bioreceptor 310 to the transducer 250.

FIG. 5 illustrates HEPES, which is an example of the zwitterionic buffer 340 included in the detection layer 300.

The FET 100 according to an embodiment of the present disclosure may be a device for detecting a biochemical molecule and is also called a bioFET.

The bioFET may refer to a device that enable high-sensitivity measurement of extremely small amounts of biochemical molecules.

In the FET (bioFET) 100 according to an embodiment of the present disclosure, a field effect may be formed by a charge (carrier) formed between the oxide layer 120 and the semiconductor substrate 110 by applying a constant voltage Vg to a gate electrode (the first gate electrode 230 and/or the second gate electrode 240). Thus, a channel connecting the source electrode 210 and the drain electrode 220 may be formed. A constant voltage Vds may be formed between the source electrode 210 and the drain electrode 220.

Therefore, the amount of charge (carrier) may be adjusted by adjusting the voltage Vg applied to the gate electrode (the first gate electrode 230 and/or the second gate electrode 240). Accordingly, the amount of current between the source electrode 210 and the drain electrode 220 may be adjusted.

In particular, according to an embodiment of the present disclosure, graphene may be used in the transducer 250, and a bioreceptor (biological sensing material) 310 can be immobilized on the graphene by a linker (320). As another example, the bioreceptor 310 may be immobilized on graphene by a self-assemble monolayer (SEM) technique.

Accordingly, a signal due to binding affinity with a molecule to be detected (target molecule) may be detected with high sensitivity by using the bioFET according to an embodiment of the present disclosure.

The FET (bioFET) 100 according to an embodiment of the present disclosure may advantageously maximize a small change above a device surface, such as a change in surface potential due to binding between the bioreceptor 310 and a biomolecule or a change in pH, to a large current change through a gating effect. To maximize this, it is advantageous to operate at a gate voltage at which a slope of a transfer curve of the FET 100 is the greatest. It may be important to select an appropriate gate voltage value. Here, the transfer curve may mean a curve of a source-drain current Ids with respect to the gate voltage Vg.

The field effect of the FET 100 may be seen through a transfer curve of a device in which gating is induced.

In general, it may be difficult to obtain a gating effect in air or gas phase in bioFETs. In a typical bioFET, gating occurs when water (or a buffer) connects a gate electrode and a transducer, but in a gas phase, it may be difficult to induce gating between electrodes unless a high voltage is applied to the gate electrode. When a high voltage is applied to the gate electrode, the stability of the device may be compromised, and problems such as signal abnormalities in the device may occur.

However, the FET (bioFET) 100 according to an embodiment of the present disclosure described above may increase transconductance through gating induction under a gaseous condition without applying a high voltage. Charge changes caused by gaseous molecules, such as olfactory receptors, may be effectively transmitted to the transducer.

The characteristics of the FET 100 are described as follows.

### 1. Voltage-Current Characteristics

The voltage-current characteristics of the FET 100 according to an embodiment of the present disclosure are as shown in Equation 1 below.

$${I}_{d s} - \frac{W}{L} μ {C}_{g} {V}_{d s} \vert {V}_{g} - {V}_{CNP} \vert \qquad [ Equation 1 ]$$

### 2. Transconductance of Transducer (Graphene Layer)

The transconductance of a transducer (graphene layer) 250 of the FET 100 according to an embodiment of the present disclosure is as shown in Equation 2 below.

That is, transconductance gm may mean a slope of a source-drain current with respect to the gate voltage V_{g}.

$${g}_{m} = \frac{W}{L} μ {C}_{g} {V}_{d s} \qquad [ Equation 2 ]$$

The meaning and unit of each symbol in Equations 1 and 2 are as follows.
V_{CNP}=charge neutral point [V]
V_{g}=gate voltage [V]
gₘ=transconductance of graphene layer [A/V]
C_{g}=gate capacitance per unit area [F/m²]
W=width of graphene's active area [m]
L=length of graphene's active area [m]
μ=electron or hole mobility of graphene layer [m²/V/s]

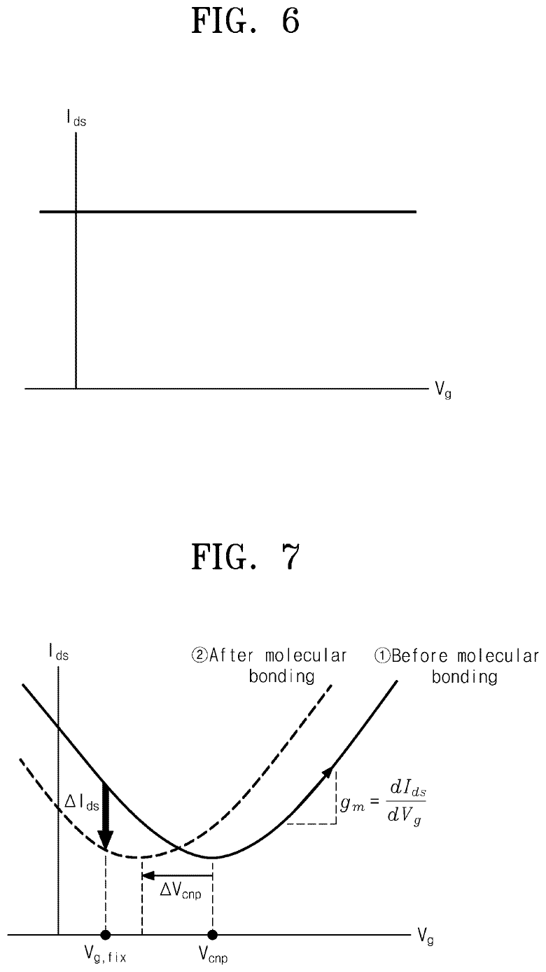

FIG. 6 is a graph showing a transfer curve of an FET in a general case. FIG. 7 is a graph showing a transfer curve of an FET according to an embodiment of the present disclosure.

In general, a transfer curve of a field effect transistor does not appear as a curve when there is no gating effect, unlike in FIG. 6. That is, in this case, even if a gate voltage Vg is applied, there is no change in a signal Ids, and thus the field effect transistor may not operate.

In the FET 100 according to an embodiment of the present disclosure, as shown in FIG. 7, when the gate voltage Vg is applied, a V-shaped transfer curve may be formed.

When a gating effect occurs as such, before the bioreceptor 310 and a sample (gas molecule) are combined, a state is as shown in curve \textcircled{1}, but after the sample (gas molecule) is combined with the bioreceptor 310, the state moves to curve \textcircled{2}, and an effect of the gate voltage Vg moving by Δ may be obtained.

Here, a direction of ΔVcnp may vary depending on a change in polarity of charge due to molecular binding with the bioreceptor 310. For example, when a change in charge is positive, Δ may decrease to negative, and when a change in charge is negative, Δ may increase to positive.

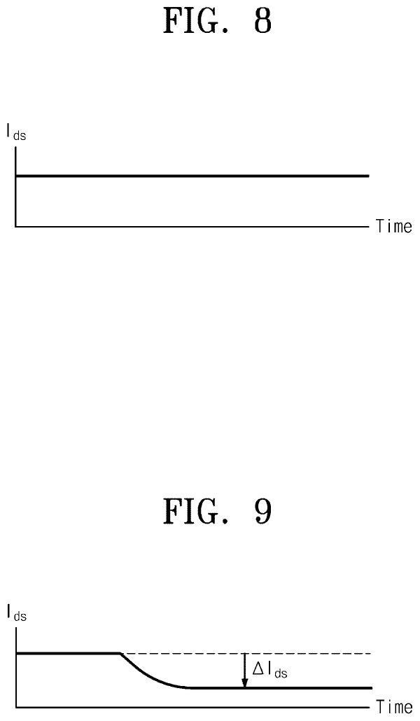

FIG. 8 is a graph showing a transfer curve of an FET over time in a general case. FIG. 9 is a graph showing a transfer curve of an FET over time according to an embodiment of the present disclosure.

In general, a transfer curve of a field effect transistor does not appear as a curve over time when there is no gating effect, unlike in FIG. 8. That is, in this case, even if a gate voltage Vg is applied, there is no change in a signal Ids, and thus the field effect transistor may not operate.

In the FET 100 according to an embodiment of the present disclosure, a gating effect may be obtained, and as shown in FIG. 7, a current change of ΔIds may occur due to a change in Vcnp of a transfer curve induced by a reaction with a sample (gas molecule).

In this case, the polarity of ΔIds may be determined by a direction of Vcnp and an applied voltage (gate voltage) Vg,fix.

Here, the meaning and unit of each symbol shown in FIGS. 6 to 9 are as follows.
ΔI_{ds}=drain current variation [V]
ΔV_{cnp}=charge neutral point variation [V]
V_{g,fix}=externally applied fixed voltage

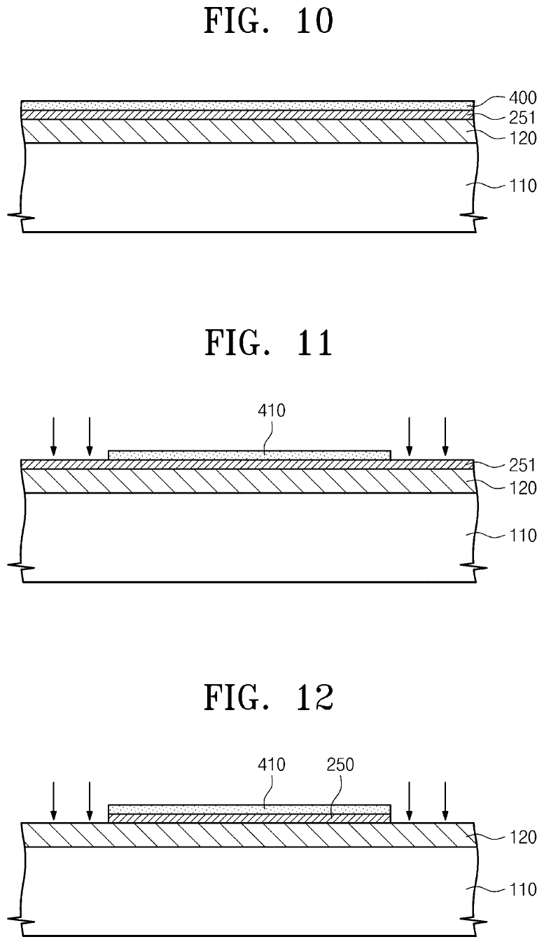

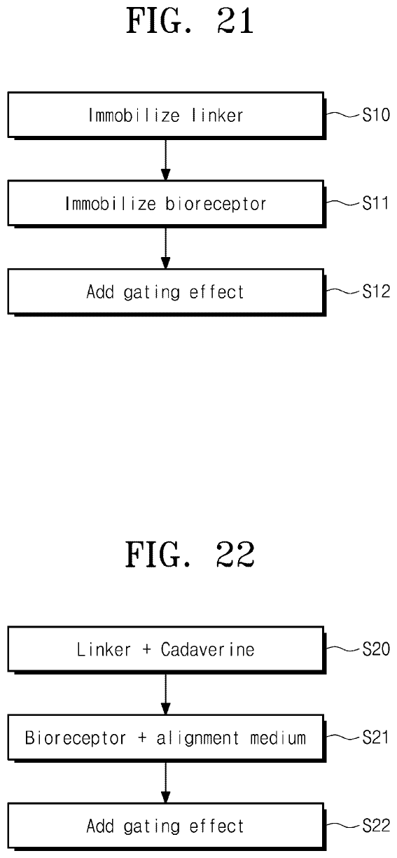

FIGS. 10 to 20 are cross-sectional views showing a manufacturing process of an FET according to an embodiment of the present disclosure. FIG. 21 and FIG. 22 are flowcharts showing a manufacturing process of a detection layer of an FET according to an embodiment of the present disclosure.

Hereinafter, with reference to FIGS. 10 to 22, a manufacturing process of the FET 100 according to an embodiment of the present disclosure will be described.

First, referring to FIG. 10, a base structure may be prepared in which a silicon oxide (SiO₂) layer 120 and a graphene layer 251 are sequentially stacked on a silicon (Si) substrate 110. The graphene layer 251 may be patterned to form the transducer 250.

A first photoresist (PR) 400 may be applied on the graphene layer 251 to pattern the graphene layer 251.

Thereafter, referring to FIG. 11, the first PR 400 may be patterned to form a patterned first PR 410.

Then, referring to FIG. 12, the graphene layer 251 may be etched using the patterned first PR 410 to form the transducer 250. Then, the patterned first PR 410 may be removed. Hereinafter, the transducer 250 is described interchangeably with the graphene layer.

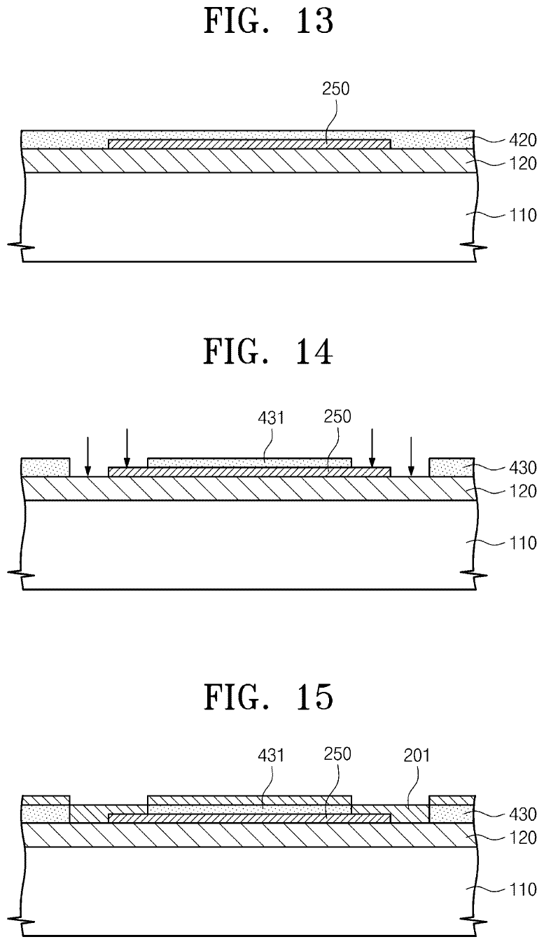

Referring to FIGS. 13 and 14, a photo process for forming an electrode may be performed. In detail, first, referring to FIG. 13, a second PR 420 may be formed in a state in which the transducer 250 is patterned on the oxide layer 120.

Then, referring to FIG. 14, patterned second PRs 430 and 431 that open portions in which electrodes (source electrode and drain electrode) are to be formed may be formed. As such, the patterned second PRs 430 and 431 may include a first portion 431 located on the graphene layer 250 with a portion (for example, both ends) of the graphene layer 250 open and a second portion 430 located at an end side of the oxide layer 120.

Referring to FIG. 15, a metal layer 201 may be formed on the patterned second PRs 430 and 431.

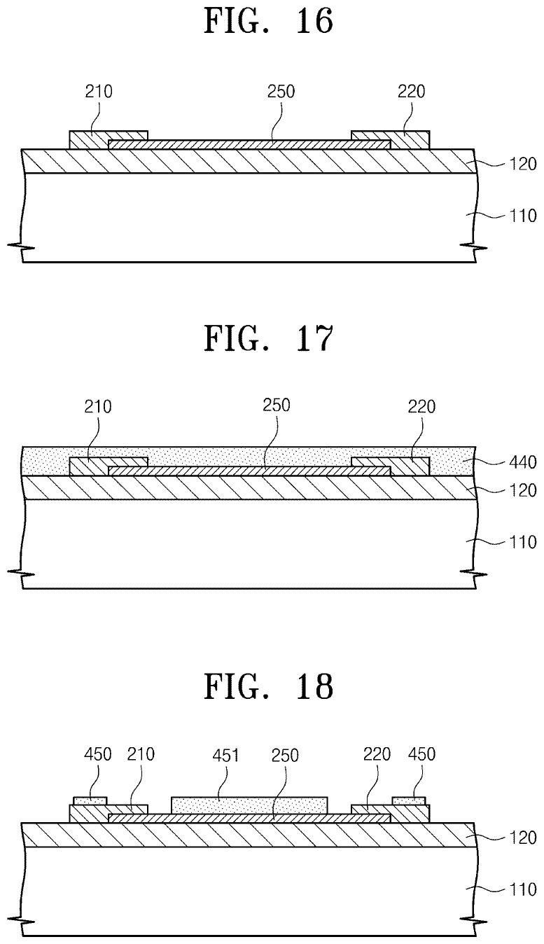

Then, when the patterned second PRs 430 and 431 are removed, a base structure may be formed in which the source electrode 210 is formed at one side of the graphene layer 250 patterned on the oxide layer 120 located on the substrate 110 and the drain electrode 220 is formed at the other side of the graphene layer 250, as shown in FIG. 16.

To summarize this manufacturing process, a process of manufacturing a base structure in which the source electrode 210 and the drain electrode 220 that are spaced apart from each other in a first direction on the oxide layer 120 located on the substrate 110 are arranged and the transducer 250 is located to connect the source electrode 210 and the drain electrode 220 to each other may be performed.

As such, in a state where the base structure is formed, operation S10 of immobilizing the linker 320 on the transducer 250 of the base structure, operation S11 of immobilizing the bioreceptor 310 on the linker 320, and operation S12 of adding a gating effect that provides transistor characteristics via coupling with at least a portion of the transducer 250 may be performed. Operation S12 of adding the gating effect may include an operation of applying the zwitterionic buffer 340.

According to an embodiment of the present disclosure, as shown in FIGS. 17 to 20, prior to the operation of forming the detection layer 300, an operation of forming an insulating layer that partially exposes an upper surface of an electrode, which include the source electrode 210 and the drain electrode 220, may be performed.

To this end, referring to FIG. 17, a third PR 440 that covers the oxide layer 120, the source electrode 210 and the drain electrode 220, and the graphene layer 250 may be formed in a state in which the source electrode 210 is formed at one side of the graphene layer 250 patterned on the oxide layer 120 and the drain electrode 220 is formed at the other side of the graphene layer 250.

Then, referring to FIG. 18, patterned third PRs 450 and 451 that open portions in which an insulating layer is to be formed may be formed. As such, the patterned third PRs 450 and 451 may include a first portion 451 located on the graphene layer 250 with a part (for example, both ends) of the graphene layer 250 open, and a second portion 450 located partially on the source electrode 210 and the drain electrode 220.

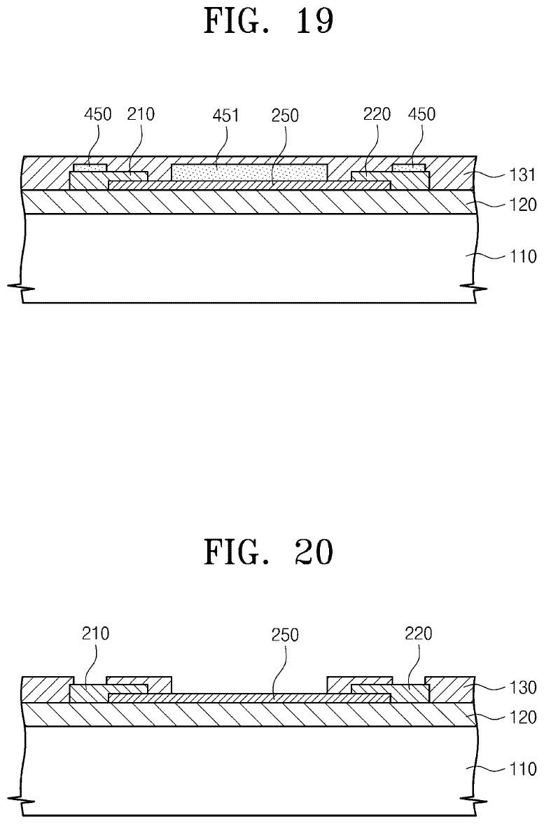

Then, referring to FIG. 19, an insulating layer 131 may be formed on the patterned third PRs 450 and 451.

Then, when the patterned third PRs 450 and 451 are removed, an insulating layer 130 that partially exposes an upper surface of the electrode including the source electrode 210 and the drain electrode 220 may be formed, as shown in FIG. 20. The insulating layer 130 may expose the graphene layer 250 therethrough. The insulating layer 130 may cover each connecting portion of the graphene layer 250 and the source electrode 210 and the drain electrode 220.

The insulating layer 130 may prevent a channel formed by the graphene layer 250 and the electrodes including the source electrode 210 and the drain electrode 220 from being electrically connected when a sample is an ionic solution.

Referring to FIG. 22, the alignment medium 330 may be used in the operation of forming the detection layer 300. As such, in the operation of using the alignment medium 330, Cadaverine may be coupled to the linker 320 while the linker 320 is immobilized on the transducer 250 having the base structure (S20).

Then, the operation of immobilizing the bioreceptor 330 may include operation S21 of adding the alignment medium 330 that is coupled to Cadaverine.

As such, Cadaverine may be coupled to the linker 320 and the alignment medium 330 may be coupled to the bioreceptor 310 to immobilize the bioreceptor 310. In this case, an immobilization rate and directionality may be improved by inducing coupling between Cadaverine and the alignment medium 330.

Then, operation S22 of adding a gating effect that provides transistor characteristics via coupling with at least a portion of the transducer 250 may be performed. Operation S22 of adding the gating effect may include an operation of applying the zwitterionic buffer 340.

The above description is provided merely by way of example to illustrate the present disclosure, and it will be understood by those of ordinary skill in the art that various modifications and changes may be made without departing from the essential features of the disclosure.

Accordingly, the embodiments disclosed herein are intended to describe, not to limit, the scope of the disclosure, and the scope of the disclosure should not be construed as being limited by these embodiments.

The scope of protection of the present disclosure shall be defined by the following claims, and all modifications or equivalents falling within the scope of the claims shall be construed as being included within the scope of the disclosure.

### INDUSTRIAL APPLICABILITY

According to the present disclosure, a field effect transistor for detecting biochemical molecules and a method of manufacturing the same may be provided.

What is claimed is:

1. A field effect transistor comprising:
a substrate including a semiconductor;
an oxide layer located on the substrate;
a source electrode and a drain electrode that are located spaced apart from each other in a first direction of the oxide layer;
a first gate electrode and a second gate electrode that are located spaced apart from each other in a second direction of the oxide layer;
a transducer located to connect the source electrode and the drain electrode;
a zwitterionic buffer that is at least partly coupled to the transducer and provides transistor characteristics;
a bioreceptor located on the transducer; and
a linker located between the transducer and the bioreceptor.

2. The field effect transistor of claim 1, wherein the transducer includes a graphene layer.

3. The field effect transistor of claim 1, wherein the transducer forms a channel between the source electrode and the drain electrode.

4. The field effect transistor of claim 1, further comprising: an alignment medium which is located between the bioreceptor and the linker.

5. The field effect transistor of claim 4, wherein the alignment medium is coupled such that the bioreceptor and the linker are arranged in series.

6. The field effect transistor of claim 1, wherein the zwitterionic buffer includes hydroxyethyl piperazine ethane sulfonicacid (HEPES).

7. The field effect transistor of claim 1, wherein the zwitterionic buffer provides a V-shaped transfer curve.

8. A field effect transistor comprising:
a substrate including a semiconductor;
an oxide layer located on the substrate;
a source electrode and a drain electrode that are located spaced apart from each other in a first direction of the oxide layer;
a first gate electrode and a second gate electrode that are located spaced apart from each other in a second direction of the oxide layer;
a transducer located to connect the source electrode and the drain electrode; and
a detection layer located on the transducer,
wherein the detection layer includes:
a bioreceptor located on the transducer; and
a zwitterionic buffer that provides transistor characteristics to the transducer.

9. The field effect transistor of claim 8, further comprising a linker located between the transducer and the bioreceptor.

10. The field effect transistor of claim 8, wherein the transducer includes a graphene layer.

11. The field effect transistor of claim 8, further comprising an alignment medium which is located between the bioreceptor and the linker.

12. The field effect transistor of claim 11, wherein the alignment medium is coupled such that the bioreceptor and the linker are arranged in series.

13. The field effect transistor of claim 8, wherein the zwitterionic buffer includes hydroxyethyl piperazine ethane sulfonicacid (HEPES).

14. The field effect transistor of claim 8, wherein the zwitterionic buffer provides a V-shaped transfer curve.

15. The field effect transistor of claim 8, further comprising an insulating layer that partially exposes upper surfaces of the source electrode and the drain electrode.

16. A method of manufacturing a field effect transistor, the method comprising:
manufacturing a base structure in which a source electrode and a drain electrode, located spaced apart from each other in a first direction, are arranged on an oxide layer located on a substrate including a semiconductor and a transductor is provided to connect the source electrode and the drain electrode;
immobilizing a linker on a transducer of the base structure;
immobilizing a bioreceptor on the linker; and
applying a zwitterionic buffer that is coupled to at least a portion of the transducer to provide transistor characteristics.

17. The method of manufacturing the field effect transistor of claim 16, wherein Cadaverine is coupled to the linker.

18. The method of manufacturing the field effect transistor of claim 16, wherein the immobilizing of the bioreceptor includes adding an alignment medium coupled to Cadaverine.

19. The method of manufacturing the field effect transistor of claim 16, wherein the transducer includes a graphene layer.

20. The method of manufacturing the field effect transistor of claim 16, wherein the zwitterionic buffer includes hydroxyethyl piperazine ethane sulfonicacid (HEPES).
